(19) United States
(12) Patent Application Publication
(10) Pub. No.: US 2004/0192995 A1
(43) Pub. Date: Sep. 30, 2004

(54) Process for recovery of sulfur from sour/natural gas using biodegradable carboxylic acid metal chelates

(75) Inventors: Aniruddha Subhash Deshpande, Pune (IN); Narendra Vasant Sankpal, Pune (IN); Bhaskar Dattatraya Kulkarni, Pune (IN)

Correspondence Address: LADAS & PARRY, 26 WEST 61ST STREET, NEW YORK, NY, 10023, US

(73) Assignee: COUNCIL OF SCIENTIFIC AND INDUSTRIAL RESEARCH

(21) Appl. No.: 10/396,108

(22) Filed: Mar. 25, 2003

(51) Int. Cl. C07C 7/00

(52) U.S. Cl. 585/864; 585/833; 585/855

(57) ABSTRACT

A process for recovery of sulfur from sour/natural gas stream containing H₂S is described. It involves contacting the stream with an aqueous solution of biodegradable water soluble carboxylic acid metal chelate. This carboxylic acid metal chelate contains an oxidizing polyvalent metal e.g. Fe (III) and one of the acid from gluconic acid, malic acid, citric acid, succinic acid, oxalic acid. The hydrogen sulfide gas is reduced to hydrogen and elemental sulfur, which is recovered as precipitate. The iron in the metal chelate is reduced in the process and can be regenerated by oxidation.

### FIELD OF THE INVENTION

This invention relates to an improved process for the recovery of sulfur from sour/natural gas. More particularly it relates to the recovery of sulfur from sour/natural gas contaminated with H₂S or a mixture of gases containing H₂S using biodegradable chelates. The reduced chelated metal mass is oxidized with air or oxygen and the precipitated elemental sulfur is separated. The regenerated chelated metal mass is recycled and reused for the conversion of H₂S to elemental sulfur.

### BACKGROUND OF THE INVENTION

In chemistry chelate or chelating agents are compounds, which form coordinate covalent bonds with a metal ion. Chelates are thus coordination compounds in which a central metal atom is bounded to two or more other atoms in at least one other molecule or ion (called ligand) such that at least one hetro-cyclic ring is formed with the metal atom as part of each ring.

Chelants are used in variety of applications including food processing, soaps, detergents, cleaning products, pharmaceuticals, pulp and paper processing, water treatment, metalworking and metal plating solutions, textile processing solutions, fertliers, animal feeds, herbicides, rubber and polymer chemistry, photofinishing, oilfield chemistry. Some of these activities result in chelants entering the environment. For instance, agricultural uses or detergent uses may result in measurable quantities of the chelants being in water. It is therefore, desirable that chelants degrade after use. Biodegradability, that is susceptibility to degradation by microbes, is particularly useful because the microbes are generally naturally present in environments. Commonly used chelant like EDTA (Ehtylenediamine tetraacetic acid) is biodegradable, but the rate is somewhat slower. It would be desirable to have a chelating agent, which degrades faster than EDTA or other commonly used chelants like HEDTA (Hydroxyethylethylene-diaminetriacetic acid), NTA (Nitrilotriacetic acid), DTPA (Diethylenetriaminepentaacetic acid), etc.

Natural gas as well as refiner gas streams are commonly contaminated with sulfur compounds, especially hydrogen sulfide. If substantial amounts of hydrogen sulfide are present, regulatory restrictions dictate that special precautions must be taken to purify the gas streams. In non-populated areas, generally a maximum of two tons per day of sulfur is allowed to be vented as sulfur oxides flare-off gas per processing plant. In populated areas even more stringent restrictions are applied.

Gas desulfurization using iron chelates is a commercial process where the liquid redox chemistry of alternate reduction and oxidation of iron complex is gainfully utilized. McManus and Martell (1997) provide a comprehensive review of this technology in the article. We shall therefore provide additional major developments since there.

C. Pagella et.al. “H₂S gas treatment by iron bioprocess”, Chemical Engineering Sciences 55(2000) 2185-2194 discussed a novel chemical-biological process based on combination of absorption with chemical reaction and biological oxidation.

C. A. Lieder has reported in U.S. Pat. No. 4,332,781 a process for the removal of H₂S gas and C₂S from gas stream in a staged procedure characerized by conversion of the H₂S to produce sulfur in aqueous solution or suspension. The reactant material includes polyvalent metal bound to monomeric chelate.

F. Engelhardt et.al. in U.S. Pat. No. 4,518,745 disclosed a number of metal chelates of water soluble copolymer, The copolymers are useful as dyestuff auxiliaries and leather retanning agents.

U.S. Pat. No. 5,741,555 discloses use of polyamino disuccinic acids for effective chelants for use in photography and gas conditioning but polyamino acids take longer time for biodegradation.

Polyamino disuccinic acids, despite possessing some chelating properties have not found wide usage. For instance, a better known member of family, namely ethylenediamine disuccinic acid (EDDS) has not been a widely used chelants. The preparation of polyamino disuccinic acids is discussed by Kezeodan et.al. in U.S. Pat. No. 3,158,635 where their use in rust removal is disclosed. Atkinson in U.S. Pat. No. 4,704,233 disclosed use of EDDS in detergents to enhance removal of organic stains and mention about its biodegradability.

U.S. Pat. No. 4,889,609 discloses a process in solution for removing H₂S from a stream of natural gas. Sodium nitrite in the solution serves as an oxidizing agent for H₂S. In absence of a catalyst, the process suffers from conversion of nitrite ion into NH₃. This conversion is suppressed by the incorporation of a transition metal chelate complex. The metal is preferably chromium, copper or iron and the chelate complex is preferably EDTA or TEA, with acceptable performance obtained from HEDTA or NTA. Use of EDTA and TEA however generate streams that are slow or non-biodegradable.

U.S. Pat. No. 5,152,970 discloses a process for removal of hydrogen sulfide from sour gas by passing the gases in the presence of oxygen and steam over a catalytic absorption mass, which contains inert porous support material and catalytically active metal sulfides and/or metal oxides. The elemental sulfur formed gets simultaneously deposited on the absorption mass, which is then regenerated. The whole process is carried out at higher temperature.

J. O. Porath in U.S. Pat. No. 4,423,158 discloses a process for introducing a chelate-forming group into a polymer such as polystyrene. The adsorbent for bivalent or multivalent metal ions so obtained are useful in ion exchange chromatography. Grinstead et.al. in U.S. Pat. No. 4,859,437 describe a cyclic continuous process for reduction of hydrogen sulfide by using organic polymeric chelate to sulfur. The process includes removal of elemental sulfur and ultrafilteration or dialysis to regenerate reduced polyvalent metal. The regeneration of polyvalent metal is expensive.

In view of the above state of the art the need for providing an improved process for the removal of sulfur from sour/natural gas using biodegradable metal chelates is clearly evident.

It is also desirable that the biodegradability of the chelate be many times more than the conventionally used chelates.

### OBJECTS OF THE INVENTION

The main object of the present invention is to provide a process for the recovery of sulfur from sour/natural gas which is eco-friendly.

It is another object of the invention to provide an improved process for the removal of sulfur from sour/natural gas waste streams using water soluble chelated metal, where hydrogen sulfide having been absorbed into an alkaline solution of chelated metal is converted to yield both elemental sulfur and hydrogen.

Another objective of the present invention is to decompose poisonous gas such as hydrogen sulfide into separate amounts of hydrogen and sulfur.

The third objective is to provide a process for the removal of sulfur from sour/natural gas using chelated metal and chelating agent which is easily biodegradable and eco-friendly.

### SUMMARY OF THE INVENTION

Accordingly the present invention provides an improved process for recovery of sulfur from sour/natural gas, which comprises contacting the sour/natural gas flow with an aqueous solution of a biodegradable water-soluble carboxylic acid metal chelate till the sour/natural gas coming out of the solution is free from H₂S, reoxidising the carboxylic metal chelate solution by passing a source of oxygen through the solution, separating the suspended form of sulfur obtained and drying to obtain sulfur.

In one embodiment of the invention, the metal chelate comprises a polyvalent metal chelatable in both oxidized and reduced states.

In a further embodiment of the invention, the polyvalent metal is selected from the group consisting of tin, lead, copper, manganese, platinum, tungsten, nickel, palladium, chromium, cobalt, vanadium, titanium, tantalum, zirconium and molybdenum.

In another embodiment of the invention the carboxylic acid metal chelate is a transition metal chelate of iron selected from the group consisting of solution of sulfates, nitrates, thiosulfates, chlorides, acetates, oxalates and phosphates.

In another embodiment of the invention, the transition metal salt is chelated with a carboxylic acid.

In another embodiment of the invention, the carboxylic acid is selected from the group consisting of mono-, di- and tri-carboxylic acids obtained from biodegradable source.

In a further embodiment of the invention, the carboxylic acid is selected from the group consisting of succinic acid, citric acid, malic acid, gluconic acid and oxalic acid.

In another embodiment of the invention, the solution is maintained at the pH of 7.0 to 9.0 using a hydroxide of an alkaline earth element selected from the group consisting of Na, K and Mg.

In another embodiment of the invention, the oxygen source is selected from air and molecular oxygen.

In another embodiment of the invention, the process for recovery of elemental sulfur is selected from a continuous stirred reactor process, bubble column reactor process, airlift type reactor process and a batch process.

In another embodiment of the invention, the concentration of the chelating agent is in the range of 500 ppm to 20000 ppm.

In another embodiment of the invention, the sour/natural gas is contacted with the metal chelate solution at ambient temperature and atmospheric pressure.

### DETAILED DESCRIPTION OF THE INVENTION

The present disclosure is directed to biodegradable aqueous carboxylic acid metal chelate oxidizer, which is used in an aqueous solution for removal of H₂S from sour/natural gas. With its signature ‘rotten egg’ smell, H₂S is not only odorous, but corrosive and neuro-toxic too. It is produced naturally, by anaerobic decomposition of sulfur-bearing materials and synthetically, by a host of chemical process operations, including hydrogenation and hydrodesulfurization and coking. Most people can detect concentration even as small as 0.01 PPM. The gas is toxic in relatively light concentrations and should be removed from natural gas as soon as possible. Many processes have been developed to convert H₂S to some innocuous forms, such as elemental sulfur and sulfates.

The sour/natural gas is introduced into an aqueous solution of biodegradable carboxylic acid metal chelate. The present process is typically carried out at room temperature and atmospheric pressure and the pH of chelated metal solution is maintained in the range 7.0 to 9.0. The sour/natural gas is bubbled through the reactor containing the solution of aqueous metal chelate.

Generally, any polyvalent metal chelatable in both oxidized and reduced states can be used as the metal component of polymeric chelate. In the present invention ferric iron salts of sulfate, nitrate, chloride are particularly preferred. The polyvalent metal chelate should be capable of oxidizing hydrogen sulfide (H₂S) while being reduced itself to the corresponding chelate of the metal in a lower valence state and should then be oxidizable by oxygen or similar oxidation means to chelate of the metal in higher valence state in a typical redox reactions. Other polyvalent metals which can be used include tin, lead, copper, manganese, platinum, tungsten, nickel, palladium, chromium, cobalt, vanadium titanium, tantalum, zirconium and molybdenum.

The chelate is preferably biodegradable carboxylic acids like gluconic acid, citric acid, succinic acid, malic acid and oxalic acid.

The solution should be preferably maintained at pH range from about 7.0 to about 9.0. The pH of the resulting iron chelate solution can be adjusted with an alkaline material such as ammonia solution, sodium carbonate or dilute caustic i.e. sodium hydroxide solution. Contact of H₂S, with the chelate takes place in the pH range from about 6 to about 10. The more preferred range of pH is from about 6.5 to about 9.0 and tie most preferred range of pH is from 7.0 to 9.0. In general highest portion of the range is preferred in order to operate at high efficiency of H₂S absorption. Since the H₂S is an acid gas, there is tendency for the H₂S to lower the pH of the aqueous alkaline solution. Lower pH is preferable in the presence of CO₂ to reduce absorption thereof. Optimum pH also depends upon stability of a particular polyvalent metal chelate. At the pH values below about 6.0 the efficiency of H₂S absorption is low so as to be generally impractical. At pH values greater than 10.0, for instance with iron as the polyvalent metal, the precipitation of insoluble iron hydroxide may occur resulting in decomposition of the iron chelate.

Any inert water-soluble biodegradable chelate capable of chelating an oxidizing polyvalent metal is suitable in the present process. “Inert”, in this context, is defined as not detrimentally reactive in the system to an intolerable extent. The carboxylic acids like citric acid, gluconic acid, malic acid, succinic acid, oxalic acid are used in combination with ferric iron salts like ferric sulfate, ferric nitrate and ferric chloride. The combination of above said each salt is made with above said each carboxylic acid in stoichiometric proportions in aqueous medium. Concentrations of iron chelate for H₂S removal is made from range about 500 PPM to 20,000 PPM. The pH of the solution is maintained at about 7.0 to about 9.0.

The present invention therefore provides an improved process for recovery of sulfur from sour/natural gas, which comprises contacting the sour/natural gas flow with an aqueous solution of a biodegradable water-soluble carboxylic acid metal chelate at a pH in the range of about 7.0 to 9.0, at atmospheric pressure till the sour/natural gas coming out of the solution is free from H₂S, reoxidising the carboxylic metal chelate solution by passing through the said solution a source of oxygen, separating the sulfur obtained in the suspended form by conventional methods and drying the sulfur by conventional methods to obtain sulfur.

The carboxylic acid metal chelate may be the transition metal chelate of iron such as solution of sulfates, nitrates, thiosulfates, chlorides, acetates, oxalates, phosphates. The transition metal salt used is chelated with carboxylic acids selected from mono-, di-, tri-carboxylic acids from biodegradable source exemplified by succinic acid, citric acid, malic acid, gluconic acid and oxalic acid.

The solution is maintained at the pH of about 7.0 to 9.0 by using hydroxides of alkaline earth elements such as Na, K and Mg.

The source of oxygen may be air or molecular oxygen. In a feature of the present invention the process used for the recovery of elemental sulfur may be a continuous stirred reactor, bubble column reactor, or airlift type reactor or a batch process.

The process of the present invention is described hereinbelow with reference to examples which are illustrative only and should not be construed to limit the scope of the present invention in any manner whatsoever.

### EXAMPLE-1

500 ml of water was placed in a container with 10 grams of ferric nitrate. On total dissolution of salt, 31.01 ml of gluconic acid solution was added to it. Add 10% extra acid for complete and proper chelation of metal salt. The pH of the solution was checked and 5N NaOH solution was added to maintain the pH in the range 7.0 to about 9.0. The forgoing was mixed at room temperature and atmospheric pressure to form true solution. The H₂S gas was then slowly bubbled through the solution at constant flow rate. While the test contents began initially as a true solution, the solution soon becomes dark in color, showing slurry of precipitated sulfur. Continue to pass the feed gas through the solution until no more precipitation of sulfur occurs. This is readily tested by passing the outlet gas through a tube containing AgNO₃ solution which begun to turn black. Pass the H₂S gas for 10 more minutes so as to ensure complete conversion of H₂S to elemental sulfur. While conversion of H₂S to elemental sulfur occurs the Fe (III) simultaneously gets reduced to Fe (II) and so to reoxidize Fe (II) to Fe (III) excess air or oxygen was sparged through solution. On oxidation of chelated solution the color of the precipitated sulfur turns to off-white. Total sulfur recovery on completion of the process was 90 milligrams of 99% purity (as evaluated using microanalysis).

### EXAMPLE-2

500 ml of water was placed in a container with concentration of ferric nitrate solution varying from 10,000 PPM to about 1000 PPM. On total dissolution of salt, gluconic acid solution was added to it in proportion. Add 10% extra acid for complete and proper chelation of metal salt. The pH of the solution was checked and 5N NaOH solution was added to maintain the pH in the range 7.0 to about 9.0. The forgoing was mixed at room temperature and atmospheric pressure to form true solution. The H₂S gas was then slowly bubbled through the solution at constant flow rate. While the test contents began initially as a true solution, the solution soon becomes dark in color, showing slurry of precipitated sulfur. Continue to pass the feed gas through the solution until no more precipitation of sulfur occurs. This is readily tested by passing the outlet gas through a tube containing AgNO₃ solution which begun to turn black. Pass the H₂S gas for 10 more minutes so as to get maximum conversion of H₂S to elemental sulfur. While conversion of H₂S to elemental sulfur occurs the Fe (III) simultaneously gets reduced to Fe (II) and so to reoxidize Fe (II) to Fe (III) excess air or oxygen was sparged through solution. On oxidation of chelated solution the color of the precipitated sulfur turns to off-white. The total sulfur recovered was of the 99% purity (as evaluated using microanalysis), in the range of 13 milligrams to 117 milligrams per gram of chelated metal.

TABLE 1

| | Amount of | | |
|---|---|---|---|
| | Gluconic acid | Milligram of | |
| Concentration in | used for | sulfur recovered | Time required for |
| PPM of chelated | chelation in | per gram of | completion of |
| Iron. | milliliter | Chelated iron. | process in min. |
| 20,000 | 32.00 | 90.00 | 80 |
| 10,000 | 16.00 | 116.83 | 48 |
| 5,000 | 8.00 | 13.29 | 45 |
| 2,000 | 3.20 | 27.7 | 50 |
| 1,000 | 1.60 | 51.00 | 50 |

### EXAMPLE-3

500 ml of water was placed in a container with concentration of ferric sulfate solution varying from 20,000 PPM to about 1000 PPM. On total dissolution of salt, malic acid solution was added to it in proportion from 9.024 grams to 0.4512 grams. Add 10% extra acid for complete and proper chelation of metal salt. The pH of the solution was checked and 5N NaOH solution was added to maintain the pH in the range 7.0 to about 9.0. The forgoing was mixed at room temperature and atmospheric pressure to from true solution. The H₂S gas was then slowly bubbled through the solution at constant flow rate. While the test contents began initially as a true solution, the solution soon becomes dark in color, showing slurry of precipitated sulfur. Continue to pass the feed gas through the solution until no more precipitation of sulfur occurs. This is readily tested by passing the outlet gas through a tube containing AgNO₃ solution which begun to turn black. Pass the H₂S gas for 10 more minutes so as to get maximum conversion of H₂S to elemental sulfur. While conversion of H₂S to elemental sulfur occurs the Fe (III) simultaneously gets reduced to Fe (II) and so to reoxidize Fe (II) to Fe (III) excess air or oxygen was sparged through solution. On oxidation of chelated solution the color of the precipitated sulfur turns to off-white. The total sulfur recovered was of the 99% purity (as evaluated using microanalysis), in the range of 25 milligrams to 84 milligrams per gram of chelated metal.

TABLE 2

| Concentration | Amount of malic | Milligram of | |
|---|---|---|---|
| in PPM of | acid used for | sulfur recovered | Time required for |
| chelated | chelation in | per gram of | completion of |
| Iron. | grams | Chelated iron. | process in min. |
| 20,000 | 9.024 | 28.20 | 40 |
| 10,000 | 4.5122 | 83.95 | 35 |
| 5,000 | 2.2561 | 66.14 | 25 |
| 2,000 | 0.9024 | 25.44 | 25 |

### EXAMPLE-4

500 ml of water was placed in a container with concentration of ferric chloride solution varying from 20,000 PPM to about 1000 PPM. On total dissolution of salt, malic acid solution was added to it in proportion from 13.6059 grams to 0.6802 grams. Add 10% extra acid for complete and proper chelation of metal salt. The pH of the solution was checked and 5N NaOH solution was added to maintain the pH in the range 7.0 to about 9.0. The forgoing was mixed at room temperature and atmospheric pressure to form true solution. The H₂S gas was then slowly bubbled through the solution at constant flow rate. While the test contents began initially as a true solution, the solution soon becomes dark in color, showing slurry of precipitated sulfur. Continue to pass the feed gas through the solution until no more precipitation of sulfur occurs. This is readily tested by passing the outlet gas through a tube containing AgNO₃ solution which begun to turn black. Pass the H₂S gas for 10 more minutes so as to get maximum conversion of H₂S to elemental sulfur. While conversion of H₂S to elemental sulfur occurs the Fe (III) simultaneously gets reduced to Fe (II) and so to reoxidize Fe (II) to Fe (III) excess air or oxygen was sparged through solution. On oxidation of chelated solution the color of the precipitated sulfur turns to off-white. The total sulfur recovered was of the 99% purity (as evaluated using microanalysis), in the range of 59 milligrams to 127 milligrams per gram of chelated metal.

TABLE 3

| | Amount of | Milligram of | |
|---|---|---|---|
| Concentration in | malic acid used | sulfur recovered | Time required |
| PPM of chelated | for chelation in | per gram of | for completion of |
| Iron. | grams | Chelated iron. | process in min. |
| 20,000 | 13.6059 | 59.88 | 21 |
| 10,000 | 6.8029 | 99.26 | 35 |
| 5,000 | 3.4014 | 101.57 | 30 |
| 2,000 | 1.30659 | 121.80 | 12 |
| 1,000 | 0.68029 | 126.48 | 10 |

### EXAMPLE-5

500 ml of water was placed in a container with concentration of ferric chloride solution varying from 20,000 PPM to about 1000 PPM. On total dissolution of salt, citric acid solution was added to it in proportion from 14.2153 grams to 0.7107 grams. Add 10% extra acid for complete and proper chelation of metal salt. The pH of the solution was checked and 5N NaOH solution was added to maintain the pH in the range 7.0 to about 9.0. The forgoing was mixed at room temperature and atmospheric pressure to form true solution. The H₂S gas was then slowly bubbled through the solution at constant flow rate. While the test contents began initially as a true solution, the solution soon becomes dark in color, showing slurry of precipitated sulfur. Continue to pass the feed gas through the solution until no more precipitation of sulfur occurs. This is readily tested by passing the outlet gas through a tube containing AgNO₃ solution which begun to turn black. Pass the H₂S gas for 10 more minutes so as to get maximum conversion of H₂S to elemental sulfur. While conversion of H₂S to elemental sulfur occurs the Fe (III) simultaneously gets reduced to Fe (II) and so to reoxidize Fe (II) to Fe (III) excess air or oxygen was sparged through solution. On oxidation of chelated solution the color of the precipitated sulfur turns to off-white. The total sulfur recovered was of the 100% purity (as evaluated using microanalysis), in the range of 40 milligrams to 72 milligrams per gram of chelated metal.

TABLE 4

| Concentration | Amount of citric | Milligram of | |
|---|---|---|---|
| in PPM of | acid used for | sulfur recovered | Time required for |
| chelated | chelation in | per gram of | completion of |
| Iron. | grams | Chelated iron. | process in min. |
| 20,000 | 14.2153 | 58.38 | 20 |
| 10,000 | 7.10765 | 44.21 | 21 |
| 5,000 | 3.5537 | 41.86 | 8 |
| 2,000 | 1.42153 | 71.52 | 39 |
| 1,000 | 0.71076 | 41.04 | 30 |

During the experimental runs it was observed that the rate of precipitation of sulfur varies with the concentration and type of metal salt and carboxylic acid used. In other words the time required for completion of process varies from case to case for otherwise similar operating conditions. Dynamic studies and rigorous optimization may further improve the yield and recovery of sulfur. In any case, the recovery and its rate are almost always better than that obtained using conventional EDTA process. Experimental runs were also conducted in continuous mode of reactor operation for a constant flow of H₂S laden gas and chelating solution. Similar quantitative results were obtained for the continuous reactor. The comparative rate of recovery for an improved biodegradability of carboxylic acid metal chelates shall prove to be useful for commercial exploitation of this process.

We claim:

1. A process for recovery of sulfur from sour/natural gas, which comprises contacting the sour/natural gas flow with an aqueous solution of a biodegradable water-soluble carboxylic acid metal chelate till the sour/natural gas coming out of the solution is free from H₂S, reoxidising the carboxylic metal chelate solution by passing a source of oxygen through the solution, separating the suspended form of sulfur obtained and drying to obtain sulfur.

2. A process as claimed in claim 1 wherein the metal chelate comprises a polyvalent metal chelatable in both oxidized and reduced states.

3. A process as claimed in claim 2 wherein the polyvalent metal is selected from the group consisting of tin, lead, copper, manganese, platinum, tungsten, nickel, palladium, chromium, cobalt, vanadium, titanium, tantalum, zirconium and molybdenum.

4. A process as claimed in claim 1 wherein the carboxylic acid metal chelate is a transition metal chelate of iron selected from the group consisting of solution of sulfates, nitrates, thiosulfates, chlorides, acetates, oxalates and phosphates.

5. A process as claim 4 wherein the transition metal salt is chelated with a carboxylic acid.

6. A process as claimed in claim 5 wherein the carboxylic acid is selected from the group consisting of mono-, di- and tri-carboxylic acids obtained from biodegradable source.

7. A process as claimed in claim 6 wherein the carboxylic acid is selected from the group consisting of succinic acid, citric acid, malic acid, gluconic acid and oxalic acid.

8. A process as claimed in claim 1 wherein the solution is maintained at the pH of 7.0 to 9.0 using a hydroxide of an alkaline earth element selected from the group consisting of Na, K and Mg.

9. A process as claimed in claim 1 wherein the oxygen source is selected from air and molecular oxygen.

10. A process as claimed in claim 1 wherein the process for recovery of elemental sulfur is selected from a continuous stirred reactor process, bubble column reactor process, airlift type reactor process and a batch process.

11. A process as claimed in claim 1 wherein the concentration of the chelating agent is in the range of 500 ppm to 20000 ppm.

12. A process as claimed in claim 1 wherein the sour/natural gas is contacted with the metal chelate solution at ambient temperature and atmospheric pressure.
